(19) United States
(12) Patent Application Publication
(10) Pub. No.: US 2005/0176096 A1
(43) Pub. Date: Aug. 11, 2005

(54) Stabilized biocatalysts and methods of bioconversion using the same

(76) Inventors: Seok-Joon Kwon, Daejon Metropolitan City (KR); Soo Keun Choi, Daejon Metropolitan City (KR); Heung Chae Jung, Daejon Metropolitan City (KR); Jae Gu Pan, Daejon Metropolitan City (KR); Joon Sung Chang, Daejon Metropolitan City (KR); Tae Jung Park, Daejon Metropolitan City (KR)

Correspondence Address: NIXON & VANDERHYE, PC, 901 NORTH GLEBE ROAD, 11TH FLOOR, ARLINGTON, VA, 22203, US

(21) Appl. No.: 10/510,744

(22) PCT Filed: Apr. 9, 2002
(86) PCT No.: PCT/KR02/00617

(51) Int. Cl. C12P 21/06; C12N 15/74; C12N 1/21

(52) U.S. Cl. 435/69.1; 435/471; 435/472; 435/252.3

(57) ABSTRACT

The present invention relates to a biocatalyst displayed on spore or virus surface and a method of bioconversion using the same, in particular to a method of bioconversion using a biocatalyst, which comprises the steps of: (a) preparing a vector for spore surface display comprising a gene construct containing a gene encoding a display motif and a gene encoding the biocatalyst, wherein, when expressed, the gene construct expresses the display motif and the biocatalyst in a fusion form and the biocatalyst is displayed on a spore surface; (b) transforming a host cell with the vector for spore surface display; (c) displaying the biocatalyst on the spore surface of the host cell; (d) recovering the spore displaying on its surface the biocatalyst; and (e) performing the bioconversion reaction using the spore displaying on its surface the biocatalyst, and a biocatalyst.

### FIELD OF THE INVENTION

The present invention relates to a stabilized biocatalyst and a method of bioconversion using the same, in particular to a stabilized biocatalyst displayed on a surface of spore or virus and a method of bioconversion using the same.

### DESCRIPTION OF THE RELATED ART

The technology of surface display in which a bacterium displays on its surface a foreign protein has a variety of applications such as bacteria vaccine, high-throughput screening of peptide and antibody library, whole cell adsorbent and whole cell biocatalyst (7). In particular, a whole cell biocatalyst prepared by expressing a biocatalyst in microorganism can provide lower-costly biocatalysts and cofactors compared to a protein biocatalyst (7).

Where a biocatalyst is expressed in a cell or a plasma membrane space, permeabilizing agents are necessary to allow substrates and products converted to pass through a cell membrane. However, according to this method, the biocatalyst is very likely to be inactivated and side reactions occur due to various enzymes in cell. Georgiou group has employed Lpp-OmpA-fused β-lactamase displayed on E. coli surface to hydrolyze β-lactam, and obtained above 50-fold reaction rate compared to that expressed in periplasm (5).

Such display technology in which enzyme or antibody is expressed on a surface of microorganism allows to avoid protein purification and immobilization processes and to yield a biocatalyst immobilized on cell surface spontaneously by culturing microbes (6). However, the whole cell biocatalyst described above cannot be continuously reused and applied to various bioconversion reactions such as reactions in organic solvents. That is because a variety of problems such as cell disruption, inactivation of biocatalyst due to protease, reduction of cell viability and detachment of biocatalyst from cell surface occur during bioconversion reaction. To overcome such problems, Georgiou group has attempted to crosslink E. coli whole cells displaying β-lactamase on their surface for increasing the stability of whole cell biocatalyst by protecting cell disruption (6). However, such attempt has been revealed unsuccessful because most of enzymes are inactivated during crosslinking process and the continuous observation on cell structure is required.

Consequently, there is a need in the art for a biocatalyst showing higher stability Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

To be free from the shortcoming of conventional biocatalysts described previously, the present inventors have made intensive researches and have found that a biocatalyst displayed on spore or virus surface allows to reuse continuously for a long period of time, to have various resistance to extreme environment and to be applicable to various bioconversion reactions. As a result, the present inventors have completed this invention.

Accordingly, it is an object of this invention to provide a method of bioconversion using a biocatalyst displayed on spore or virus surface.

It is another object of this invention to provide a biocatalyst displayed on a spore surface by fusing covalently to a display motif.

It is still another object of this invention to provide a biocatalyst displayed on a spore or virus surface by virtue of noncovalent bonds.

In an aspect of this invention, there is provided a method of bioconversion using a biocatalyst, which comprises the steps of: (a) preparing a vector for spore surface display comprising a gene construct containing a gene encoding a display motif and a gene encoding the biocatalyst, wherein, when expressed, the gene construct expresses the display motif and the biocatalyst in a fusion form and the biocatalyst is displayed on a spore surface; (b) transforming a host cell with the vector for spore surface display; (c) displaying the biocatalyst on the spore surface of the host cell; (d) recovering the spore displaying on its surface the biocatalyst; and (e) performing the bioconversion reaction using the spore displaying on its surface the biocatalyst.

The principle of the present invention lies in the improvement of biocatalyst stability and workability by means of displaying and immobilizing a biocatalyst on a spore surface by using recombinant DNA technology and microbial spore exhibiting stability to extreme environment such as high temperature, radioactivity, toxin, high (osmotic) pressure, acid, base, dry and organic acid.

The researches on bioconversion using microbial spore so far known are as follows: bioconversion in column reactor with Bacillus spores immobilized on polyacryamide (18); and bioconversion of perfumes such as geraniol and nerol by using Aspergillus and Penicillum spores (3). However, two researches employ an enzyme existed in spore per se and no display method of foreign enzyme by using recombinant DNA technology.

U.S. Pat. No. 5,766,914 discloses a method of producing and purifying enzymes using fusion protein between CotC or CotD among spore coat proteins of Bacillus subtilis and lacZ as reporter. However, as disclosed, the activity of enzyme expressed has been very low and the display of enzyme on spore surface has never been demonstrated by means of reliable methods such as biochemical, physical and immunological methods. In addition to this, the inner coat protein, CotD is enclosed by outer coat protein of 70-200 nm thickness, which makes it difficult to be exposed to spore surface. In case of fusion protein expression using outer coat protein, CotC, the activity of enzyme is increased by four-fold in comparison with that of CotD; however, the activity, 0.02 U, is considered negligible, in particular, in consideration of industrial scale. Furthermore, the patent document reports that the thermal stability of biocatalyst immobilized on spore and biocatalyst in a free form is not different. Therefore, as recognized from the disclosure of the patent document, the invention is not considered to employ a display system on spore surface; furthermore, the recognition and understanding of the inventors on advantages of a display system on spore surface is not found. In other words, the patent is not directed to a display system on spore surface and a bioconversion.

In contrast, the biocatalyst of the present invention displayed on spore surface exhibits enhanced stability, e.g., thermal stability and stability to organic solvent compared to a biocatalyst in free form.

The present inventors have been recognized the shortcomings of conventional technologies described above and to overcome the shortcomings, researched on a surface display with spore coat in terms of enzymatic, immunological and physiochemical approaches. As a result, the present inventors have accomplished to establish the best surface display system. The invention of the surface display system has been filed for patent application under the number of PCT/KR01/02124, the teachings of which are incorporated herein by reference. Furthermore, based on the display system on spore surface, the present inventors have developed a bioconversion system capable of performing a bioconversion with higher efficiency even under extreme environment.

According to one embodiment of this invention, the display motif is a spore coat protein. Where the spore coat protein is used as a display motif, the gene encoding spore coat protein is derived from a spore-forming Gram negative bacterium including Myxococcus; a spore-forming Gram positive bacterium including Bacillus; a spore-forming Actionmycete; a spore-forming yeast including Saccharomyces cerevisiae, Candida and Hansenulla or a spore-forming fungus, but not limited to. More preferably, the gene encoding spore coat protein is derived from a spore-forming Gram positive bacterium, most preferably, Bacillus including Bacillus subtilis and Bacillus polymyxa, etc.

The term “display motif” used herein refers to a molecule for displaying a biocatalyst on a spore surface expressed in a spore in a fusion form with a biocatalyst. This term is interchangeably used with the term “expression motif”. Therefore, the term “display” or “expression” is used with referring to the term “surface”, there is no intended distinction between them.

The gene of spore coat protein useful in this invention includes cotA, cotB, cotC, cotD (W. Donovan et al., J. Mol. Biol., 196:1-10(1987)), cotE (L. Zheng et al., Genes & Develop., 2:1047-1054(1988)), cotF (S. Cutting et al., J. Bacteriol., 173:2915-2919(1991)), cotG, cotH, cotJA, cotJC, cotK, cotL, cotM, cotS, cotT (A. Aronson et al., Mol. Microbiol., 3:437-444(1989)), cotV, cotW, cotX, cotY, cotZ (J. Zhang et al., J. Bacteriol., 175:3757-3766(1993)), spoIVA, spoVID and soda, but not limited to. According to a preferred embodiment, the gene of spore coat protein is cotA, cotE, cotF, cotG, cotH, cotJA, cotJC, cotK, cotL, cotM, cotS, cotT, cotV, cotW, cotX, cotY, cotZ, spoIVA, spoVID or soda, more preferably, cotE or cotG and most preferably, cotG.

The spore coat proteins used in this invention circumvent a necessity for passage across cell membrane, so that they need no secretion signal and target signal, thereby ensuring a surface display of protein such as β-galactosidase in orderly fashion which is difficult to pass across cell membrane.

While a naturally-occurring gene is used as the gene of spore coat protein, it is preferred that the gene modified for having more compatibility for spore surface display is used. The modified form of the gene is obtained by DNA shuffling method (Stemmer, Nature, 370: 389-391(1994)), StEP method (Zhao, H., et al., Nat. Biotechnol., 16: 258-261 (1998)), RPR method (Shao, Z., et al., Nucleic acids Res., 26: 681-683 (1998)), molecular breeding method (Ness, J. E., et al., Nat. Biotechnol., 17: 893-896 (1999)), ITCHY method (Lutz S. and Benkovic S., Current Opinion in Biotechnology, 11: 319-324 (2000)), error prone PCR (Cadwell, R. C. and Joyce, G. F., PCR Methods Appl., 2: 28-33 (1992)), point mutagenesis (Sambrook et al., Molecular Cloning: A Laboratory Manual, Cold Spring Harbor, N. Y., 1989), nucleotide mutagenesis (Smith M. Annu. Rev. Genet. 19: 423-462 (1985)), combinatorial cassette mutagenesis (Wells et al., Gene 34: 315-323 (1985)) and other suitable random mutagenesis. Furthermore, the gene encoding spore coat protein is modified to having a substituted promoter for its promoter to enhance spore surface display. The promoter for enhancing surface display, for example, includes the promoters of cotE or cotG genes, which show higher expression level.

According the present method, as fusing a gene of coat protein and a gene of biocatalyst, the overall sequence, fragments, two or more repeated sequences of the coat protein gene are useful. In two or more repeated sequences, the repeated sequences may be the same or different each other. The overall sequence, two or more repeated sequences of the biocatalyst gene are also useful in the fusion sequence. In two or more repeated sequences, the repeated sequences may be the same or different each other. Other combinations also are useful in the fusion sequence.

It is understood by one skilled in the art that the gene construct may exist as plasmid in host cell independently or as integrated form into chromosome of host cell. Additionally, in the gene construct, it is recognized by one skilled in the art that the gene of coat protein may be followed or preceded by the biocatalyst gene. Integrated form into the counterpart gene may be useful.

It is recognized by one skilled in the art that the expression of the fusion protein between coat protein and biocatalyst can be induced by virtue of promoters of coat protein gene or biocatalyst or other suitable promoters inducible in host cell.

In one embodiment of this invention, the biocatalyst expressed in a fusion form with spore coat protein includes multimer (homomultimer and heteromultimer) as well as monomer. The surface display of multimeric proteins has been rarely reported, for instance, the surface display of alkaline phosphatase in E. coli, has resulted in the display toward the inner part of cell outer membrane (Stathopoulus et al., Appl Microbilo Biotechnol. 45(1-2) :112-119(1996)). β-galactosidase used as biocatalyst in Examples of the present invention must form a tetramer to exhibit its activity and has not been published to be successful in surface display. β-galactosidase generally cannot pass across cell membrane and comprises an amino acid sequence detrimental to cell membrane, as a result, the fusion protein between surface display motif and β-galactosidase has been recognized not to be displayed on cell surface. Therefore, the successful construction of the biocatalyst comprising spore surface-displayed β-galactosidase exemplified in Examples proves to be surprising. Further to this, the present invention is applicable to monomeric or multimeric biocatalyst that permits to provide an active bioconversion system.

Alternatively, the surface motif useful in this invention is derived from randomly-synthesized peptides. The procedures for obtaining a molecule with a desired property from randomly-synthesized peptides can be generally embodied by means of a method for screening peptide specifically bound to antibody from a phage library expressing on its surface random peptides (Scott J. K. and Smith G. P., Science, 249:386-390(1990)). For example, to screen a peptide motif capable of binding to spore surface as this invention, a phage peptide library and spores are mixed and phages bound to spores are eluted, followed by repeatedly amplifying in E. coli, so that the phage showing highest binding strength is given. Such approach is referred to as “biopanning”. According to the approach, the surface motif of interest can be obtained from randomly-synthesized peptides.

According to another embodiment of this invention, the surface motif useful in this invention is a peptide or polypeptide selected from a natural-occurring random library. The procedures for obtaining a peptide or polypeptide with a desired property from natural-occurring random library can be generally embodied in accordance with the process using a phage peptide library described above. In this case, a library containing naturally-occurring random DNA fragments is used rather than randomly-synthesized peptides. Thereafter, the random peptide library obtained thus may be displayed on a phage surface. Such approach has not been reported but may be considered successful by using a technology for purifying DNA from soil, sea water or lake water (Rondon M R et al., Cloning the soil metagenome: a strategy for accessing the genetic and functional diversity of uncultured microorganisms. Appl Environ Microbiol. 66(6) :2541-2547(2000)) and a technology for phage surface display (Scott J. K. and Smith G. P., Science, 249:386-390(1990)). According to this invention, the biocatalyst fused to display motif may include any enzyme known to one skilled in the art, for example, including a hydrolase, an oxidoreductase, a transferase, a lyase, an isomerase and a ligase. According to a preferred embodiment, the biocatalyst is a transferase that has a significant usefulness in industry, more preferably, an enzyme catalyzing transglycosylation, most preferably, β-galactosidase, levansucrase, dextransucrase, inulosucrase, glycogen synthase, chitin synthease, starch synthease, cyclomaltodextrin glucanotransferase or 4-α-glucanotransferase.

In the present bioconversion system, the fusion form of the display motif and the biocatalyst has a non-limited order to the extent that the biocatalyst is displayed on spore surface, including an order of the display motif—the biocatalyst and the biocatalyst-the display motif.

While the biocatalyst displayed spore surface according to the present invention exhibits excellent stability, it is preferred that the biocatalysts displayed on spore surface are covalently crosslinked. The covalent crosslinking could enhance stability of biocatalyst. The covalent crosslinking occurs between spore or virus surface and the biocatalyst; or between the biocatalysts. The covalent crosslinking may be formed by any method known to one skilled in the art, for example, including a chemical method such as glutaraldehyde treatment (DeSantis G. and Jones J. B. Curr. Opin. Biotechnol. 10:324-330(1999)), a physical method such as ultraviolet treatment (Graham L., and Gallop P. M. Anal. Biochem. 217:298-305(1994)), or a biochemical method such as enzyme treatment (Gao Y., and Mehta K., J. Biochem. 129:179-183(2001)). The biocatalyst subject to the treatment for crosslinking exhibits improved stability to rigorous environment such as thermal stability and is very likely to prevent the access of protease to inhibit its hydrolysis due to enzyme.

The host cell useful in this invention includes, but not limited to, a spore-forming Gram negative bacterium such as Myxococcus; a spore-forming Gram positive bacterium such as Bacillus; a spore-forming Actionmycete; a spore-forming yeast and a spore-forming fungus. Preferably, the host cell is the spore-forming Gram positive bacterium and more preferably, Bacillus. In particular, Bacillus subtilis is advantageous in the senses that genetic knowledge and experimental methods on its spore forming as well as culturing method are well known.

According to a preferred embodiment, the spore exhibits lower protease activity or no protease activity.

In the biocatalyst of this invention, the spore may be reproductive or non-reproductive, preferably, non-reproductive. For instance, non-reproductive Bacillus subtilis lack of cwlD gene is preferably used in this invention.

In the present method, the transformation of a host cell with a vector for spore surface display may be performed according to a variety of methods known to one skilled in the art. For example, where a host cell is prokaryote, e.g., Bacillus subtilis, natural transformation (C. R. Harwood, et al., Molecular Biological Methods for Bacillus, John Wiley & Sons, New York, p.416(1990)) is useful; where a host cell is eukaryote, e.g., yeast, electroporation (Becker, D. M. et al., Methods Enzymol. 194:182-187(1991)) is useful.

According a preferred embodiment, the recovery of spore is performed in such a manner that the display of the biocatalyst on the spore surface is maximized by controlling a culture period, after which culturing is terminated and the spore is then recovered. Suitable culture period may be varied depending upon the type of cell used, for example, in case of using Bacillus subtilis as host, the culture period of 16-25 hours is preferred.

In the present method, the recovery of spore may be carried out according to the conventional methods known to one skilled in the art, more preferably, renografin gradients methods (C. R. Harwood, et al., “Molecular Biological Methods for Bacillus.” John Wiley & Sons, New York, p.416(1990)).

The biocatalyst displayed on spore surface according to the present methods can be demonstrated with a wide variety of methods as follows: According to the first approach, a primary antibody is bound to the biocatalyst displayed on spore surface and then reacted with a secondary antibody labeled with fluorescent chemical to stain the spore, followed by observation with fluorescence microscope or analysis with flow cytometry. In the second approach, the biocatalyst displayed on spore surface is treated with protease, followed by measurement of the activity of the biocatalyst or detecting lower signal with fluorescence microscope or flow cytometry. In the third approach in which the biocatalyst uses a substrate with higher molecular weight, the direct measurement of the activity of the biocatalyst can provide the level of display since the substrate cannot pass across outer coat of spore.

In the present method, the bioconversion could be performed in various reaction systems, for example, water system, organic solvent system, water-organic solvent two-phase system and supercritical system. The reasons to carry out bioconversion in various reaction systems are: (a) the drawbacks associated with the technology using whole cell immobilized such as cell disruption and reduced cell viability can be overcome; (b) the spore displaying on its surface the biocatalyst can be existed in organic solvent phase due to its hydrophobicity, thereby providing improved bioconversion in water-organic solvent two-phase system; and (c) the resistances to rigorous environment such as heat, organic solvent, high pressure, dry, toxin, acid and base are significantly elevated by covalently immobilizing the biocatalyst on spore surface.

Where industrial-useful chiral compounds, precursors for drug, bio-surfactants are synthesized, it is usually advantageous that the reaction with a biocatalyst is carried out in organic solvent system or water-organic solvent system (11, 20). In such case, a biocatalyst showing high reaction rate and stability in organic solvent is particularly advantageous. Okahata group in Japan has reported that the biocatalyst coated with lipid shows higher reaction rate and stability in organic solvent (19). In addition, there is another report that β-galactosidase coated with lipid existed in organic solvent layer makes it possible to carry out transgalactosylation in water-organic solvent two-phase system that does not occur with β-galactosidase itself (17). Since the bioconversion of this invention exhibits improved stability and activity in both organic solvent and water-organic solvent two-phase systems, it is particularly suitable in producing substances with high value as described previously.

In anther aspect of this invention, there is provided a method of bioconversion using a biocatalyst, which comprises the steps of: (a) transforming a host cell harboring a genetic carrier selected from the group consisting of spore and virus with a vector containing a gene encoding the biocatalyst; (b) culturing the transformed host cell and expressing the biocatalyst in the host cell; (c) allowing to form noncovalent bonds between the expressed biocatalyst and a surface of the genetic carrier so that the biocatalyst is displayed on the surface of the genetic carrier; (d) recovering the genetic carrier displaying on its surface the biocatalyst; and (e) performing the bioconversion reaction using the genetic carrier displaying on its surface the biocatalyst.

The present inventors have been developed a method for surface-displaying with no need for a display motif. Further to this, the present inventors have found that the novel surface-displaying method is capable of surface-displaying a protein such as biocatalyst with maintaining its inherent structure and when displaying in excess, the genetic carrier maintains its viability and resistance to surrounding environment. The novel surface display system has been filed for patent application under the number of PCT/KR02/00059, the teachings of which are incorporated herein by reference.

In describing the novel surface-displaying method applied to this invention, the term “noncovalent surface-displaying method” is used herein for distinction from other methods.

The term used herein, “genetic carrier” refers to an organism displaying on its surface a biocatalyst and having the following properties: (1) selected from the group consisting of spore and virus; (2) having capacity of forming noncovalent bonds to a biocatalyst of interest with a desired dissociation constant, expressed in host cell harboring the genetic carrier; and (3) if necessary, its surface properties is able to be modified via genetic engineering method. Furthermore, in describing the noncovalent surface-displaying method applied to this invention, the term used herein “host cell” has a different meaning from one disclosed and indicated in prior publications related to surface display of protein. The term “host cell” in the noncovalent surface-displaying method refers to a cell expressing a biocatalyst of interest and having the following properties: (1) being capable of being transformed with a gene encoding a biocatalyst of interest; (2) being capable of harboring genetic carrier such as spore and virus and proliferating the genetic carrier; and (3) being capable of being manipulated genetically, if necessary. As described above, in the present bioconversion using the noncovalent surface-displaying method, the genetic carrier displaying on its surface a biocatalyst and the host cell expressing a biocatalyst have strictly different meanings.

The noncovalent surface-displaying method applied to the present bioconversion has been developed based on a novel concept, which is largely different from the conventional surface display methods. The display method takes advantage of properties of constituents on surface of genetic carrier and, in particular, noncovalent bonds between a protein on surface of genetic carrier and a biocatalyst. The principle strategy of this display, using a spore as genetic carrier, is illustratively exemplified in FIG. 10. Referring to FIG. 10, a host cell is transformed with vector carrying a sequence encoding a biocatalyst, the biocatalyst is expressed intracellularly or extracellularly at or prior to the period of forming spore and the surface display of biocatalyst is finally accomplished by virtue of noncovalent bonds between the biocatalyst and the surface of spores formed in host cell.

As described above, the striking feature of the noncovalent surface-displaying method lies in eliminating a need of a motif for surface display which is essential in conventional methods for surface display of protein. Because the method circumvents a necessity for a motif for surface display, the biocatalyst found to be difficult to pass across cell membrane, when expressed in host cell, can be displayed well on surface of genetic carrier and when host cells are lysed to expose the genetic carrier, the genetic carriers displaying on its surface the biocatalyst can be recovered. The recovered complex between biocatalyst and genetic carrier has a broad application.

The common descriptions found in the method using a display motif described previously are omitted in order to avoid the complexity of this specification leading to undue multiplicity. For example, the descriptions relating to useful spores and enzymes, transformation of host cell, recovery of spore or genetic carrier and bioconversion system are common.

According to the present method, a spore or virus can be employed as a genetic carrier. The spore is a preferable genetic carrier. When a virus is used as a genetic carrier, it is preferred to use bacteriophage, and the biocatalyst expressed in prokaryotic host cell is surface-displayed via nonconvalent bonds to coat proteins of the bacteriophage (e.g., coat proteins III or VIII in M13 bacteriophage). Where the bacteriophage is located in periplasm of host cell, the signal peptide may be fused to the biocatalyst to permit secretion toward periplasm, thereby ensuring a surface display. If the biocatalyst of interest cannot be naturally bound to coat proteins of bacteriophage, it may be fused to a motif capable of binding to coat proteins of bacteriophage in order to allow surface display.

According to a preferred embodiment, the genetic carrier has a surface protein modified to enhance noncovalent bond with the biocatalyst. The method for modification of the genetic carrier includes: (i) fusing oligopeptide or polypeptide, which enhance noncovalent bond between the biocatalyst and genetic carrier, to the surface protein of genetic carrier; (ii) subjecting the surface protein of genetic carrier to site-directed mutagenesis; and (iii) subjecting the surface protein of genetic carrier to random mutagenesis, but not limited to.

According to a preferred embodiment, the biocatalyst to be surface-displayed may be modified so as to enhance noncovalent bonds to genetic carrier. The modification methods include: (i) deleting a portion of amino acids of the biocatalyst; (ii) fusing oligopeptide or polypeptide, which enhance noncovalent bond between the biocatalyst and genetic carrier, to the biocatalyst or deleted form of (i); (iii) subjecting the biocatalyst to site-directed mutagenesis; and (iv) subjecting the biocatalyst to random mutagenesis, but not limited to. The method of deleting a portion of amino acids of the biocatalyst may be performed in various manners, for example, by deleting ionic amino acids from N-terminal sequence (e.g. signal peptide) of the biocatalyst. The biocatalyst thus modified enhances hydrophobic interaction with genetic carrier and therefore, can be surface-displayed with lower dissociation constant. It has been reported that the spore surface carries anionic charge. Therefore, it is preferred that a cationic peptide is fused to the biocatalyst for surface display.

In still another aspect of this invention, there is provided a biocatalyst displayed on a spore surface and fused covalently to a display motif.

In further aspect of this invention, there is provided a biocatalyst displayed on a spore or virus surface by virtue of noncovalent bonds.

Since the present biocatalyst is prepared during performing the present methods described above, the descriptions for the present biocatalyst are also found in those for the present methods. Therefore, the common descriptions are omitted in order to avoid the complexity of this specification leading to undue multiplicity.

### BRIEF DESCRIPTION OF THE DRAWINGS

FIG. 1 shows a genetic map of recombinant vector;

FIG. 2 represents the results of flow cytometry analysis showing β-galactosidase displayed on spore surface;

FIG. 3 shows a standard curve for the quantification of protease activity;

FIG. 4 represents thermal stability of ⊖-galactosidase in a free form (▾) and β-galactosidases displayed on spore surface of Bacillus subtilis WB700 (●) and DB104 (◯);

FIG. 5 illustrates schematically transglycosylation in water-organic solvent two-phase system by use of β-galactosidases displayed on spore surface;

FIG. 6 represents the transglycosylation efficiency of β-galactosidase in a free form (▾) and β-galactosidases displayed on spore surface (▪) in water-ethyl ether two-phase system;

FIG. 7A shows the results from TLC analysis of products in organic solvent layer after 24-hr transgalactosylation; lane 1, β-galactosidase in a free form; lane 2, β-galactosidases displayed on spore surface;

FIG. 7B shows the results from TLC analysis of products in water layer after 24-hr transgalactosylation; lanes 1-3 correspond to lactose, glucose and galactose standards, respectively; lane 4, β-galactosidases displayed on spore surface; lane 5, β-galactosidase in a free form;

FIG. 8 represents the reaction efficiency after the reuse of β-galactosidases displayed on spore surface;

FIG. 9 represents thermal stability of β-galactosidases displayed on spore surface (▪) and its crosslinked form (▴);

FIG. 10 illustrates schematically the principle of spore surface-display of biocatalyst via noncovalent bonds; and

FIG. 11 shows a genetic map of recombinant vector pCry1p-LacZ.

The following specific examples are intended to be illustrative of the invention and should not be construed as limiting the scope of the invention as defined by appended claims.

### EXAMPLES

### Example I

### Spore Surface-Display of Biocatalysts Fused to Coat Protein of Microbial Spore

I-1: Construction of Vector for Spore Surface-Display and Expression Thereof

A gene construct having the gene encoding a fusion protein between β-galactosidase and CotG protein which has been selected by the present inventors as the most appropriate coat protein for spore surface display among coat proteins consisting of spore (see PCT/KR01/02124), was constructed as follow:

To begin with, the DNA was extracted from the Bacillus subtilis 168 strain provided from Dr. F. Kunst (ATCC 23857) (13) by Kalman's method (9). The isolated DNA was served as template and 5′-primer (gcctttggatccagtgtccctagctccgag) and 3′-primer (aaaagacgtcgactttgtatttctttttgacta) of cotG were used for PCR. Taq polymerase purchased from Boehringer Mannheim was used for total 35 cycles of PCR under the condition of denaturation for 30 sec at 94° C., annealing for 30 sec at 55° C. and extension for 1 min at 72° C.

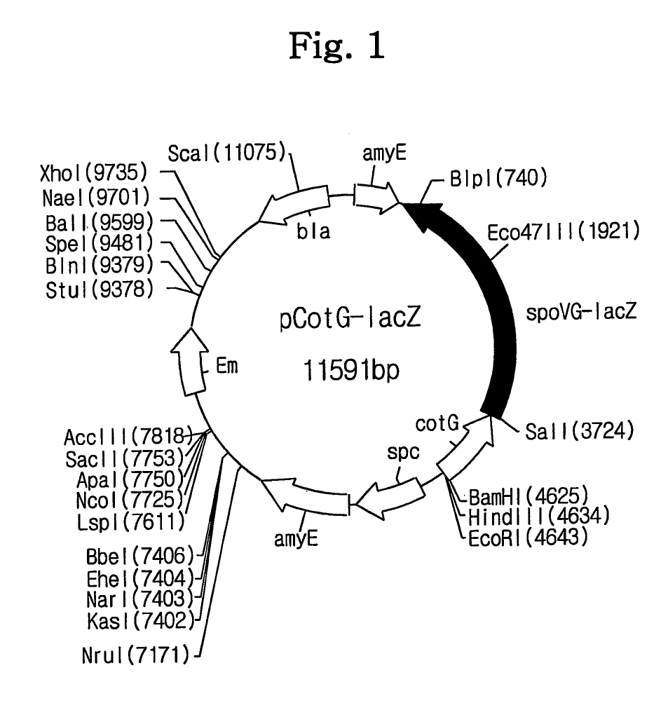

After then, each amplified PCR products were digested with BamHI and SalI and cloned between BamHI and SalI sites of plasmid pDG1728 (8) which is a gratuitous gift by Dr. P. Stragier, thereby obtaining the constructed vector to express the fusion protein of coat protein and J-galactosidase. FIG. 1 shows a genetic map of pCotG-lacZ expressing the fusion protein of CotG protein and β-galactosidase.

Constructed recombinant expression vectors were separately transformed into Bacillus subtilis DB104 (10) and WB700 (25) using natural transformation (C. R. Harwood, et al., Molecular Biological Methods for Bacillus, John Wiley & Sons, New York, p.416 (1990)).

Other methods such as conjugation or trnasduction can be applied for introduction of the recombinant vectors into Bacillus strain.

Subsequently, each Bacillus strain harboring in its chromosome the fused gene between coat protein and β-galactosidase was cultured for 24 hr at a shaking incubator (37° C., 250 rpm) in GYS medium ((NH₄)₂SO₄ 2 g/l, Yeast extract 2 g/l, K₂HPO₄ 0.5 g/l, glucose 1 g/l, MgSO₄.H₂O 0.41 g/l, CaCl₂.2H₂O 0.08 g/l, MnSO₄.5H₂O 0.07 g/l)), and the only pure spores were isolated using renografin gradients method (C. R. Harwood, et al., “Molecular Biological Methods for Bacillus.” John Wiley & Sons, New York, p.416(1990)).

The activity of each of β-galactosidase displayed on isolated spore surface and β-galactosidase in a free form was measured at 30° C. and pH,. 7.5 with 2-nitrophenyl β-D-galactolipase as substrate. For the hydrolysis activity of β-galactosidase, 1 unit was defined as the amount of the enzyme to hydrolyze 1 μmol of the substrate for 1 min. In this experiment, the activity of β-galactosidase displayed on isolated spore surface was 5 units per 1 mg of spores.

I-2: Verification of Surface Display of β-Galactosidase by Using Flow Cytometry

Flow cytometry analysis was performed to verify whether surface-displayed β-galactosidase was authentically displayed on the surface of spores as follows:

Firstly, the primary antibody obtained from the rabbit serum inoculated with β-galactosidase was bound to the β-galactosidase displayed on the surface of the spore purified in Example I-1. Then, the secondary antibody conjugated with a fluorescein, FITC was bound to the primary antibody. Using spores not containing vectors for surface display as a control, flow cytometry (FACSort, Becton Dickinson, USA) analysis was performed.

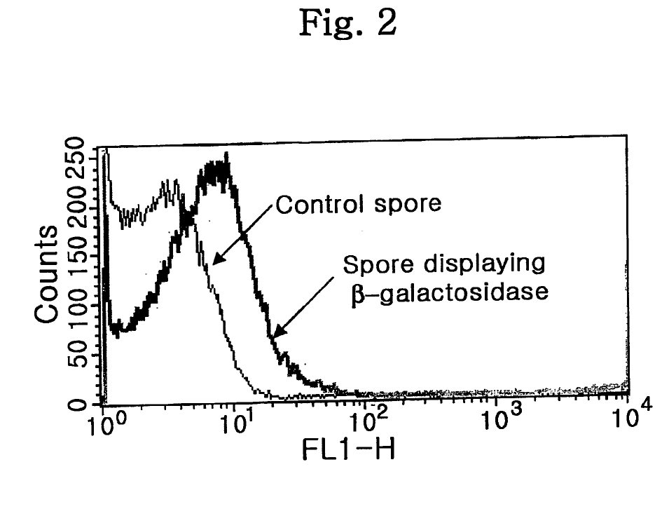

The transformed strains in the Example I-1 were inoculated to 1% of the final concentration into GYS liquid medium for sporulation and incubated at 37° C. over 24 hr. The medium was recovered and then, only the pure spores were isolated using renografin gradients method. For preventing non-specific binding, 100 μl of phosphate buffered saline (PBS) containing 3% of skim milk were added to block the pure spores. Ten μl of primary antibody (primary antibody obtained from the serum of a rabbit inoculated with β-galactosidase) was treated. Then, 10 μl of secondary antibody (Daco, Denmark, Cat No.:F0205) labeled with fluorescein, FITC (fluorescein isothiocyanate) was incubated and the reactant was washed more than 3 times with PBS. With flow cytometry (FACSort, Becton Dickinson, USA), the spores displayed with β-galactosidase were identified (FIG. 2).

In FIG. 2, the left peak corresponds to the control containing no β-galactosidase and the right peak to the spores displaying β-galactosidase. As shown in FIG. 2, it was observed that β-galactosidases expressed were successfully displayed on the surface of the spores.

While the above results relates to the surface display of β-galactosidase, it would be recognized by those skilled in the art that a variety of biocatalysts as well as β-galactosidase could be surface-displayed with referring to the methods described in this specification and the prior arts. Furthermore, the above results propose that the bioconversion system could be provided by convenient microorganism culture leading to immobilization of biocatalyst on the spore surface without the tedious efforts for immobilizing biocatalysts on carriers.

### Example II

### Comparison of Stability Between Biocatalyst Displayed on the Surface of Spores and Biocatalyst in Free Form

II-1: Stability of Biocatalyst Displayed on Spore Surface Against Protease

While the activity of the enzymes present in Bacillus spores used in the above Example is low in comparison with that in vegetative cell, they still retain most of activities as in vegetative cell (18). Particularly, the protease present in spore is likely to degrade the biocatalysts displayed on the surface of spore, so it decreases biocatalyst stability. Therefore, the inventors measured precisely the protease activity in spores and attempted to remove such protease activity.

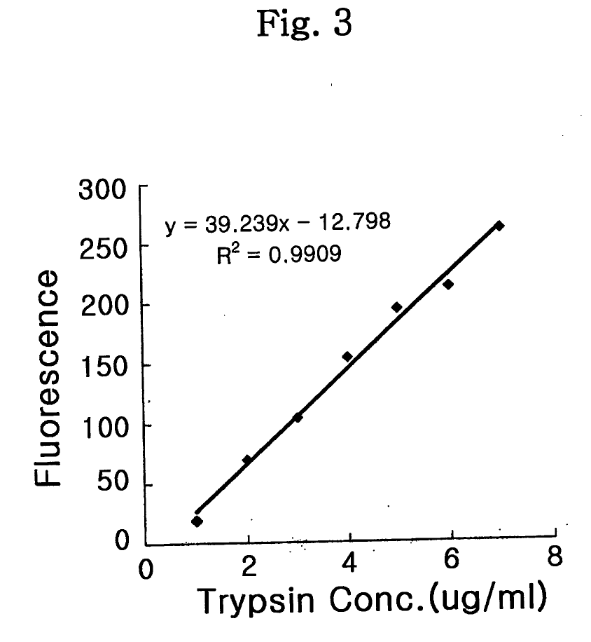

Protease activity was measured with EnzChek® Protease assay kit (Molecular probes). This approach permits to detect the fluorescence to occur through protease-catalyzed hydrolysis of casein labeled with fluorescein. Standard curve was obtained using trypsin available from Sigma (FIG. 3). For measuring the amount of proteases present in spores, DB104 strain lack of neutral and alkaline protease and WB700 strain lack of 7 proteases among proteases extracellularly secreted from Bacillus subtilis were transformed with the pCotG-lacZ expression vector according to natural transformation method as described in Example I-1.

The spores of each transformed strain were isolated, suspended to O.D of 1.7 with Tris HCl-buffer solution (pH 7.8) and the protease activity was measured (Table 1) Tris HCl-buffer solution was used as a control.

TABLE 1
Activity of Protease Depending on Type of Spore

| Spore | Concentration of protease (ug/ml) |
|---|---|
| Control | 0.0 |
| DB104 | 2.8 |
| DB104 + PMSF | 0.8 |
| WB700 | 1.4 |
| WB700 + PMSF | 0.0 |

As shown in Table 1, when 1 mM of phenylmethylsulfonylfluoride (PMSF) as protease inhibitor was treated to WB700 strain lack of 7 proteases, no protease activity was detected. Therefore, it could be appreciated that the stability of biocatalyst could be improved by virtue of displaying biocatalyst on the surface of WB700 whose the protease activity was inhibited, as such display on WB700 prevents deactivation of biocatalyst by protease.

II-2: Thermal Stability of Biocatalyst Displayed on Spore Surface

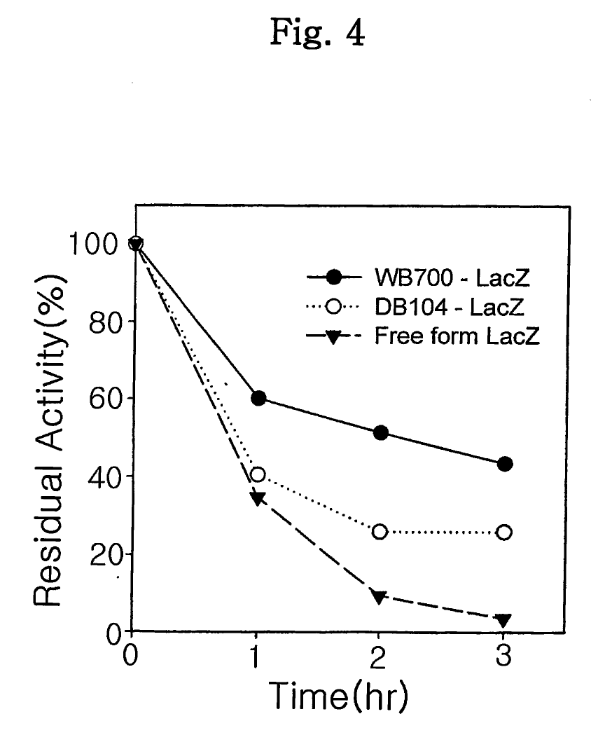

Thermal stability of β-galactosidase in a free form and β-galactosidase displayed on spore surface in DB104 or WB700 was compared. β-galactosidase in a free form and β-galactosidase displayed on spore surface were separately added to phosphate buffer (pH 7.5). With stirring at 40° C., a sample was taken at a time interval and the activity of β-galactosidase in each sample was measured according to the method in Example I-1 (see FIG. 4). β-galactosidase in a free form was obtained by expressing β-galactosidase gene used in spore display in E. coli. For a free β-galactosidase obtained, its protease activity in 1 mM PMSF was measured by the method in the above Example. Protease activity was not detected.

As shown in FIG. 4, thermal stability of the biocatalyst displayed on spore surface has been improved as compared to that of free biocatalyst (▾), and the biocatalyst displayed on the surface of WB700 (●) spores which have decreased protease activity relatively to DB104 has higher thermal stability than that of DB104 (◯).

II-3: Stability of Biocatalyst Displayed on Surface of Spores in Organic Solvent

For measuring stability in organic solvent, each of β-galactosidase in a free form and β-galactosidase displayed on surface of Bacillus spores was dispersed in 500 μl of phosphate buffer (pH 7.5) containing 1 mM PMSF, and the same volume of the various solvents described in Table 2 was added to the resultant, followed by mixing for 37 for 1 hr. The remained enzymatic activity was determined by the method described in Example I-1 (see Table 2).

TABLE 2
Stability in Organic Solvent

| Organic | Relative β-galactosidase activity | | |
|---|---|---|---|
| solvent | Free form | DB104 | WB700 |
| Control | 100.0 | 100.0 | 100.0 |
| Hexane | 84.3 | 100.0 | 100.0 |
| Ether | 48.2 | 77.2 | 97.0 |
| Toluene | 4.2 | 51.9 | 78.8 |
| Ethylacetate | 0.1 | 9.6 | 10.6 |
| Acetonitrile | 0.0 | 0.8 | 8.3 |
| Ethanol | 0.0 | 0.0 | 0.0 |

As shown in Table 2, the displayed β-galactosidase shows higher stability than that of free form β-galactosidase in various organic solvents. In addition, the biocatalyst displayed on the surface of WB700 spores which exhibit relatively decreased protease activity in comparison with DB104 shows increased stability in organic solvent.

As described above, since various methods for bioconversion in organic solvent have been studied and industrialized, the requirement for biocatalyst stable in organic solvent is greatly increased. This Example demonstrates that the displaying of biocatalyst on the surface of spores permits to improve the stability of the biocatalyst in extremely unfavorable environments such as high temperature and organic solvent, and the spores modified to have decreased protease activity is preferred.

The improved stability against various factors examined in this Example (protease, heat and organic solvent) is applicable not only to β-galactosidase, but also, to other biocatalysts displayed on spore surface for example, hydrolase such as lipase, protease and cellulase, oxidoreductase, transferase, lyase, isomerase, ligase and the like. Such conclusion is reasonable since all of biocatalysts listed above are proteins and affected by protease.

### Example III

### Bioconversion Using Biocatalysts Displayed on Spore Surface

In this Example, bioconversion with β-galactosidase displayed on spore surface was performed as a model reaction. Such bioconversion is not limited to β-galactosidase, but, is applicable to various biocatalysts displayed on spore surface, for example, hydrolase such as lipase, protease and cellulase, oxidoreductase, transferase, lyase, isomerase, ligase and the like.

III-1: Bioconversion Using β-galactosidase Displayed on Spore Surface in Water System

β-galactosidase is an enzyme hydrolyzing lactose to glucose and galactose in water system. This enzyme is generally employed for preparation of low lactose-containing milk in the food industry. In the process employing bioconversion for food preparation, preventing the microbial contamination is so critical that heat-resistance biocatalyst is required for that purpose (21). Therefore, screening and isolating heat-resistance β-galactosidase from thermophilic microorganism have been done (22). The results in Example II showed that thermal stability of biocatalyst displayed on spore surface was improved as compared to that of free biocatalyst on bioconversion in water system, and this biocatalyst immobilized on spore surface may be more advantageous for water system bioconversion at high temperature such as in Example II.

III-2: Bioconversion Using β-Galactosidase Displayed on Spore Surface in Water-Organic Solvent Two-Phase System

Forte associated with glycosylation by biocatalyst is the capability of formation of site-specific glycosidic linkage without protection/de-protection step (1, 26). This enzyme generally requires cofactors and is not conventionally available. However, the employment of a glycosidase as hydrolase for glycosylation may overcome the shortcomings mentioned above. For the formation of glycosidic linkage with hydrolase, there are suggested such methods as (a) induction of reverse hydrolysis in non- aqueous system in which the water content keeps as minimum as possible (14) and (b) transglycosylation by substitution with alcohol as receptor instead of the hydrolysis of glycosidic linkage by water (2,15). Where useful nonionic surface active agents are prepared in accordance with the above methods, the reaction is very likely to proceed in water-organic solvent two-phase system because among the substrates one (sugar) is hydrophilic and the other (alcohol) hydrophobic. However, since the organic solvent allows to inactivate enzyme and the glycosidic linkage formed is susceptible to hydrolysis by water, it is difficult to obtain higher yield. Thus, it is necessary to avoid the inactivation of glycosidase in organic solvent and the hydrolysis of glycosidic linkage formed for higher glycosylation yield.

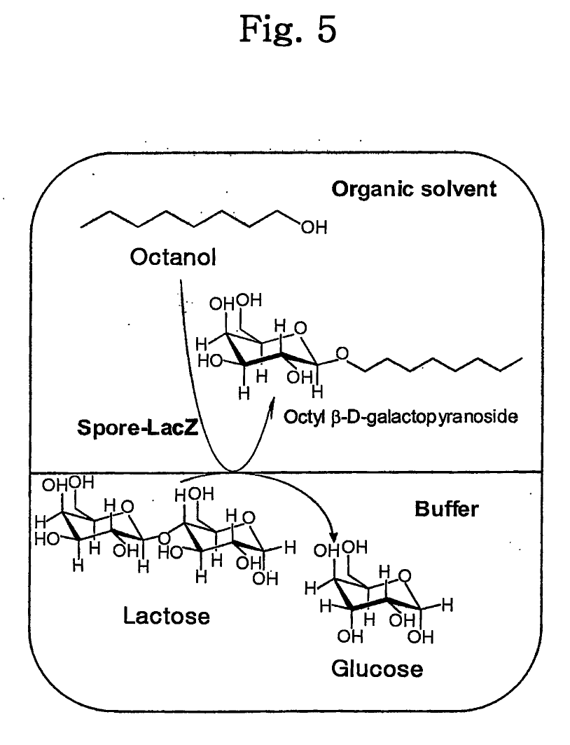

This example demonstrates that β-galactosidase displayed on spore surface permits to provide novel transglycosylation system with increased glycosylation yield (see FIG. 5). As shown in FIG. 5, in the glycosylation system provided by this invention, hydrophobic spore displaying biocatalysts and hydrophobic substrate (alcohol) are present in organic phase but hydrophilic substrate (lactose) is present in water phase. Location of the biocatalyst in organic phase ensures the hydrolysis of lactose and glycoside formed to be prevented.

This Example exemplifies that the enzyme stability in organic solvent and the glycosylation yield in water-organic solvent two-system could be increased by displaying β-galactosidase on hydrophobic Bacillus spore. For examining transglycosylation with β-galactosidase in two-phase system, octyl-β-D-galactopyranoside synthesis with lactose and octanol were performed as a model.

At first, 500 μl of ethyl ether containing 1 M octanol was added to 200 μl of phosphate buffer (pH 7.0) containing 100 mM lactose, and the mixture was agitated to form a water-organic solvent two-phase system. Thereafter, each of β-galactosidase in a free form and β-galactosidase displayed on Bacillus spore surface was added and incubated at 25° C. Two types of β-galactosidase had the same activity (5 U measured by the activity analysis of Example described above).

To determine conversion rate on time course, a sample was taken from the organic phase at a time interval and analyzed with HPLC. Mobile phase was the mixture of acetonitrile and water at the ratio of 1:1 (v:v) and the flow rate was 0.8 ml/min. Octadecylsilica column (TOSOH, Japan) was used. Standard curve was prepared using pure octyl-β-D-galactopyranoside and the concentration of reaction product was quantified with refractive index analyzer (model 133, Gilson, France).

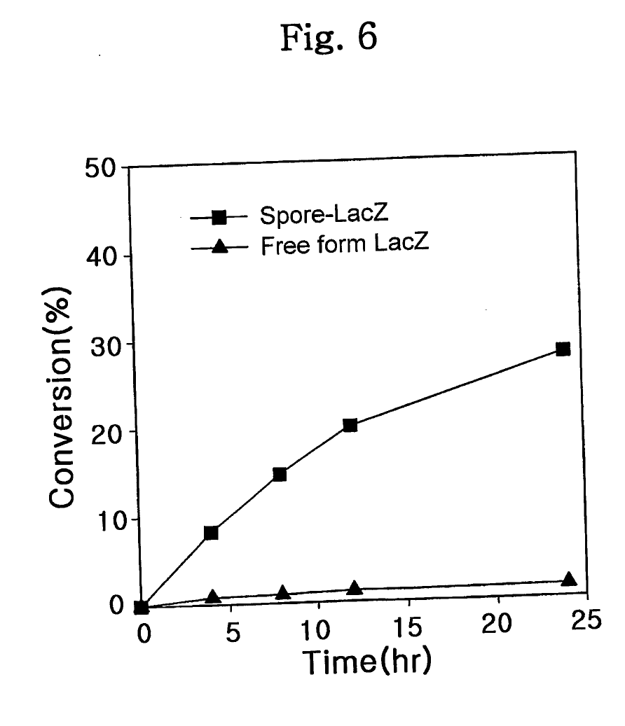

In results, the synthesis of octyl-β-D-galactopyranoside was observed in the reaction with β-galactosidase displayed on spore surface (▪), but there was no glycoside linkage formation with β-galactosidase in a free form (▴) (see FIG. 6.). The reason for this result is that β-galactosidase in a free form is likely to be degraded at interface between water and organic solvent (17).

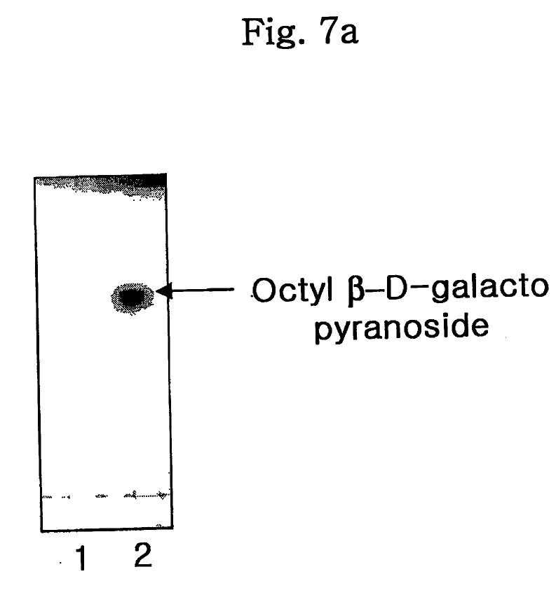

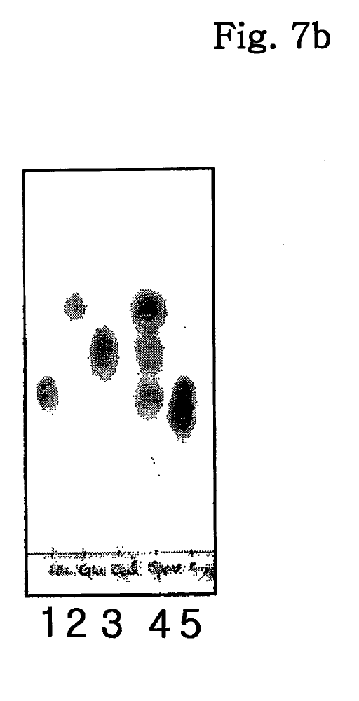

Also, after incubation for 24 hr, reactants and products present in water and organic solvent phase were analyzed with TLC (FIGS. 7a and 7b). The developing solvent was methylenechloride:methanol:water=80:15:2 (v:v:v) for analysis of organic phase, and 1-butanol:pyridine:water=6:4:1 (v:v:v) for analysis of organic phase. As similar to the results in FIG. 6, it was observed that only in the reaction with β-galactosidase displayed on spore surface, octyl-β-D-galactopyranoside was produced in organic solvent phase (see FIG. 7a). In addition, from the analysis of water phase, it was observed that any lactose was not hydrolyzed with β-galactosidase in a free form, while lactose was converted into glucose and galactose with β-galactosidase displayed on spore surface. Further, as the amount of glucose was much higher than that of galactose in water phase, it was demonstrated that little or no lactose was hydrolyzed and most of galactoses were transglycosylated with octanol (see FIG. 7b).

Octyl-β-D-galactopyranoside generated was purified through silica gel 60 column (methylene chloride:methanol:water=80:15:2, Merck, Germany), analyzed by using ¹H NMR (300 MHz) and ¹³C NMR (75.4 MHz) and its molecular weight was measured with ESI-MS. The results are summarized in Table 3.

TABLE 3
¹H NMR and ¹³C NMR Analysis of Octyl-β-D-
galactopyranoside

| | ¹³C NMR | ¹H NMR |
|---|---|---|
| 1 | 105.0 | 4.4 (1H, d, 7.2) |
| 2 | 72.6 | 3.5 (1H, dd) |
| 3 | 75.1 | 3.6-3.8 (1H, m) |
| 4 | 70.3 | 3.9 (1H, m) |
| 5 | 76.6 | 3.6-3.8 (1H, m) |
| 6 | 62.5 | 3.6-3.8 (2H, m) |
| 1′ | 70.8 | 3.6-3.8 (1H, m) 3.9 (1H, m) |
| 2′ | 33.0 | 1.6 (2H, m) |
| 3′ | 30.8 | 1.3 (2H, m) |
| 4′ | 30.6 | 1.3 (2H, m) |
| 5′ | 30.4 | 1.3 (2H, m) |
| 6′ | 27.1 | 1.3 (2H, m) |
| 7′ | 23.7 | 1.3 (2H, m) |
| 8′ | 14.4 | 0.9 (3H, t) |

As known from the results of Table 3, it is revealed that carbon 1 of galactose is reacted with octanol to maintain glycoside h-linkage. In addition, m/z values of molecular spectrum were shown 315.3 (M+Na)⁺ and 607.5 (2M+Na)⁺.

The reactions using various hydrophobic solvents in water-organic two-phase system exhibited various activities, indicated in Table 4.

TABLE 4
Influence of Organic Solvents on
Transglycosylation Using β-galactosidase Displayed on
Spore Surface in Two-Phase System

| Organic solvent | Polarity | Conversion rate (%) |
|---|---|---|
| Hexane | 0.1 | 2.8 |
| Toluene | 2.4 | 1.4 |
| Methylene Chloride | 3.1 | 19.3 |
| Ethyl ether | 2.8 | 27.2 |
| Ethyl acetate | 4.4 | 0.0 |

As described in Table 4, ethyl ether shows the highest conversion rate. In the case that the transglycosylation without solvent was performed using octanol as solvent that is a reaction substrate, the conversion rate was revealed above 30%.

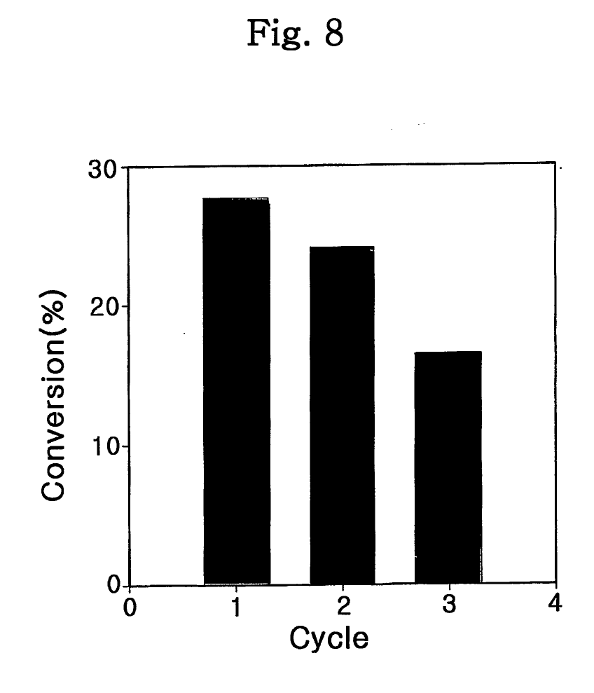

It could be understood that β-galactosidase displayed on spore surface is able to be reused due to its improved stability to organic solvent. The synthesis of octyl-β-D-galactopyranoside in two-phase system using β-galactosidase displayed on spore surface was performed up to three cycles for 12 hr each cycle, and the 59.6% of the initial activity was measured to remain (see FIG. 8).

III-3: Bioconversion in Organic Solvent System Using β-Galactosidase Displayed in Spore Surface

Enzymatic reaction in organic solvent system permits to proceed with a minimum amount of water to maintain active conformation of enzyme and to prevent the hydrolysis of products synthesized, thereby resulting in synthesis with higher yield.

For performing bioconversion in organic solvent system by use of biocatalyst displayed on spore surface, the reaction was carried out in ethyl ether solvent system using spores displaying β-galactosidase on their surface with referring to the synthesis of 5-phenyl-1-pentyl-β-D-galactopyranoside as model reaction.

2-nitrophenyl β-D-galactopyranoside (3 mg) and excess 5-phenyl-1-pentanol (>100 mg) were used as reaction substrate and ethyl ether (0.5 ml) was used as organic solvent. To the mixture containing the substrates and solvent, 2-3% phosphate buffer (pH 7.0) was added and β-galactosidase in a free form or β-galactosidase displayed on spore surface of Bacillus were added, followed by allowing the reaction at 25° C. to occur. Two types of β-galactosidase had the same activity (5 U measured by the activity analysis of Example described above).

After 48-hr reaction, β-galactosidase in a free form exhibited 23.7% of yield and β-galactosidase displayed on spore surface showed 69.8% of yield. This result is ascribed to the fact that β-galactosidase displayed on spore surface exhibits improved stability to organic solvent compared to β-galactosidase in a free form.

### Example 3

### Covalent Corsslinking of Biocatalysts Displayed on Spore Surface

To improve stability of biocatalysts displayed on spore surface, the covalent crosslinking was performed. β-galactosidases displayed on spore surface were treated with glutaraldehyde to generate crosslinks. β-galactosidases displayed on spore surface (3-6 mg/ml) washed three times with PBS were added with agitation to 0.25% (v/v) glutaraldehyde in PBS solution. All procedures were carried out at a temperature of 4° C. Following 12 hr-reaction, to the resultant was added glycine to a concentration of 100 mM for terminating crosslinking reaction, followed by centrifugation and washing with PBS, finally obtaining crosslinked spores.

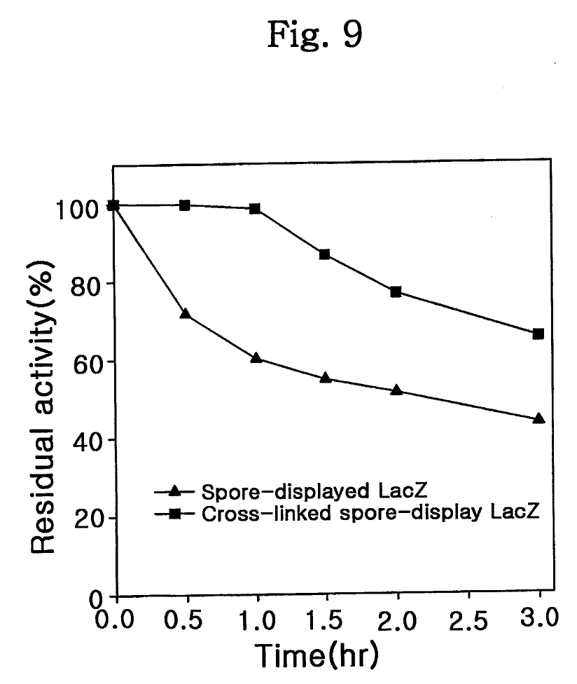

The thermal stability of crosslinked and non-crosslinked spores was examined. The test for thermal stability was performed as described above in Example. Crosslinked spores were revealed to show much higher thermal stability than non-crosslinked spores (see FIG. 9). This result is ascribed to covalent crosslinks generated to improve stability of proteins and protect the access of protease to β-galactosidase (23, 24).

### Example 4

### Display of Biocatalyst on Spore Surface via Noncovalent Bonds and Bioconversion Using the Same

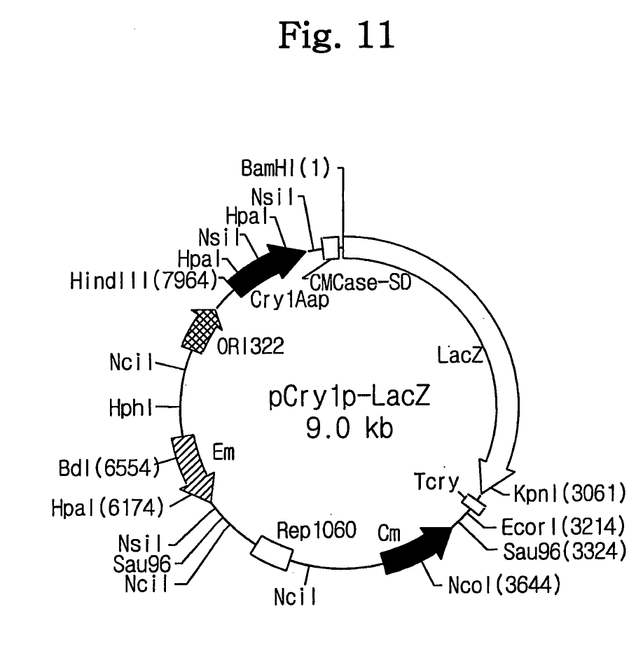

In this Example, for displaying biocatalyst on spore surface, the novel display method in which a biocatalyst with a free form is expressed in a cell and then spore coat protein and biocatalyst are linked via noncovalent bonds was employed (see FIG. 10). The novel display method is disclosed in PCT/KR02/00059 filed by the present applicant, the teachings of which are incorporated herein by reference.

PCR amplification was performed using as template pDG1728 (8) described in Example I and as primer, 5′-primer of lacZ (cgggatccgtggaagttactgacgtaag) and 3′-primer (ggggtaccgggcccttatttttgacaccagaccaactg). Taq polymerase purchased from was used for total 30 cycles of PCR under the condition of denaturation for 30 sec at 94° C., annealing for 30 sec at 55° C. and extension for 3 min at 72° C.

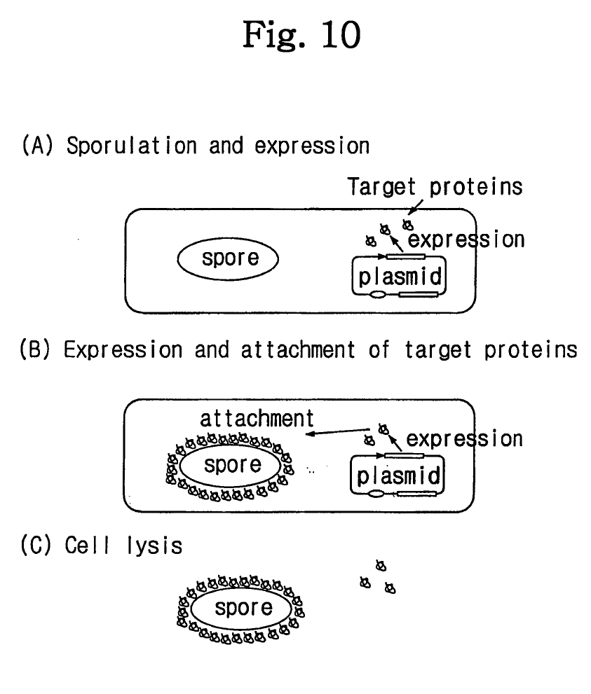

Then, each amplified PCR products were restricted with BamHI and KpnI and cloned into pCry1P-hp plasmid generated by digestion of pCry1P-CMCase-hp with BamHI and KpnI, thereby obtaining pCry1P-LacZ vector (FIG. 11). The pCry1P-LacZ vectors were transformed into Bacillus subtilis DB104 by the method described in Example I and the transformed Bacillus was cultured in GYS medium. Thereafter, the only pure spores were isolated using renografin gradients method and the measurement of β-galactosidase activity in the isolated spores was carried out. Spore surface-displaying was verified with flow cytometry.

In addition, the transglycosylation was performed as disclosed in Example III by use of the biocatalyst displayed via noncovalent bonds on spore. In two-phase system using ether as solvent, the biocatalyst showed 24% of conversion rate to octyl-β-D-galactopyranoside.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Auge, C., and D. H. Crout. 1997. Chemoenzymatic synthesis of carbohydrates. Carbohydr Res 305(β-4):307-12.
2. Crout, D. H., and G. Vic. 1998. Glycosidases and glycosyl transferases in glycoside and oligosaccharide synthesis. Curr Opin Chem Biol 2(1):98-111.
3. Demyttenaere, J., M. del Carmen Herrera, and N. De Kimpe. 2000. Biotransformation of geraniol, nerol and citral by sporulated surface cultures of Aspergillus niger and Penicillium sp. Phytochemistry 55:363-73.
4. Driks, A. 1999. Bacillus subtilis spore coat. Microbiol Mol Biol Rev 63(1):1-20.
5. Francisco, J. A., C. F. Earhart, and G. Georgiou. 1992. Transport and anchoring of beta-lactamase to the external surface of Escherichia coli. Proc Natl Acad Sci U S A 89(7):2713-7.
6. Freeman, A., S. Abramov, and G. Georgiou. 1996. Fixation and stabilization of Escherichia coli cells displaying genetically engineered cell surface proteins. Biotechnol Bioeng 52:625-30.
7. Georgiou, G., H. L. Poetschke, C. Stathopoulos, and J. A. Francisco. 1993. Practical applications of engineering gram-negative bacterial cell surfaces. Trends Biotechnol 11(1):6-10.
8. Guerout-Fleury, A. M., N. Frandsen, and P. Stragier. 1996. Plasmids for ectopic integration in Bacillus subtilis. Gene 180(1-2):57-61.
9. Kalman, S., K. L. Kiehne, J. L. Libs, and T. Yamamoto. 1993. Cloning of a novel cryIC-type gene from a strain of Bacillus thuringiensis subsp. galleriae. Appl Environ Microbiol 59(4):1131-7.
10. Kawamura, F., and R. H. Doi. 1984. Construction of a Bacillus subtilis double mutant deficient in extracellular alkaline and neutral proteases. J Bacteriol 160(1):442-4.
11. Kibanov, A. M. 2001. Improving enzymes by using them in organic solvents. Nature 409(6817):241-6.
12. Klibanov, A. M. 1986. CHEMTECH 16:354.
13. Kunst, F., N. Ogasawara, I. Moszer, A. M. Albertini, G. Alloni, V. Azevedo, M. G. Bertero, P. Bessieres, A. Bolotin, S. Borchert, R. Borriss, L. Boursier, A. Brans, M. Braun, S. C. Brignell, S. Bron, S. Brouillet, C. V. Bruschi, B. Caldwell, V. Capuano, N. M. Carter, S. K. Choi, J. J. Codani, I. F. Connerton, A. Danchin, and et al. 1997. The complete genome sequence of the gram-positive bacterium Bacillus subtilis. Nature 390(6657):249-56.
14. Ljunger, G., P. Adlercreutz, and B. Mattiasson. 1994. Enzymatic synthesis of octyl-beta-D-glucoside in octanol at controlled water activity. Enzyme Microb Technol 16:751-55.
15. Lopez, R., and A. Fernandez-Mayoralas. 1994. Enzymatic beta-galactosidation of modified monosaccharides: study of the enzymatic selectivity for the acceptor and its application to the synthesis of disaccharides. J Org Chem 59:737-45.
16. MacManus, D. A., and E. N. Vulfson. 2000. Regioselectivity of enzymatic glycosylation of 6-O-acyl glycosides in supersaturated solutions. Biotechnol Bioeng 69(6):585-90.
17. Mori, T., S. Fujita, and Y. Okahata. 1997. Transglycosylation in a two-phase aqueous-organic system with catalysis by a lipid-coated beta-D-galactosidase. Carbohydr Res 298(1-2):65-73.
18. Murata, K. 1993. Use of microbial spores as a biocatalyst. Critical Reviews in Biotechnology 13(3):173-93.
19. Okahata, Y., and T. Mori. 1997. Lipid-coated enzymes as efficient catalysts in organic media. Trends in Biotechnology 15:50-54.
20. Schmid, A., J. S. Dordick, B. Hauer, A. Kiener, M. Wubbolts, and B. Witholt. 2001. Industrial biocatalysis today and tomorrow. Nature 409(6817):258-68.
21. Shimada, A., M. Odate, N. Koyama, K. Hashino, K. Asada, and I. Kato. 1998. U.S. Pat. No. 5,744,345.
22. Takase, M., and K. Horikoshi. 1995. U.S. Pat. No. 5,432,078.
23. Tor, R., Y. Dror, and A. Freeman. 1989. Enzyme stabilization by bi-layer ‘encagement.’. Enz. Microb. Technol. 11:306-12.
24. Weiner, C., M. Sara, and U. B. Sleytr. 1994. Novel protein A matrix prepared from two dimensional protein crystals. Biotechnol. Bioeng. 43:321-30.
25. Ye, R., J. H. Kim, B. G. Kim, S. Szarka, E. Sihota, and S. L. Wong. 1999. High-level secretory production of intact, biologically active staphylokinase from Bacillus subtilis. Biotechnol Bioeng 62(1):87-96.
26. Zeng, X., R. Yoshino, T. Murata, K. Ajisaka, and T. Usui. 2000. Regioselective synthesis of p-nitrophenyl glycosides of beta-D-galactopyranosyl-disaccharides by transglycosylation with beta-D-galactosidases. Carbohydr Res 325(2) :120-31.

1-56. (canceled)

57. A method of bioconversion using a biocatalyst, which comprises the steps of:
(a) preparing a vector for spore surface display comprising a gene construct containing a gene encoding a display motif and a gene encoding the biocatalyst, wherein, when expressed, the gene construct expresses the display motif and the biocatalyst in a fusion form and the biocatalyst is displayed on a spore surface;
(b) transforming a host cell with the vector for spore surface display;
(c) displaying the biocatalyst on the spore surface of the host cell;
(d) recovering the spore displaying on its surface the biocatalyst; and
(e) performing the bioconversion reaction using the spore displaying on its surface the biocatalyst.

58. A method of bioconversion using a biocatalyst, which comprises the steps of:
(a) transforming a host cell harboring a genetic carrier selected from the group consisting of spore and virus with a vector containing a gene encoding the biocatalyst;
(b) culturing the transformed host cell and expressing the biocatalyst in the host cell;
(c) allowing to form noncovalent bonds between the expressed biocatalyst and a surface of the genetic carrier so that the biocatalyst is displayed on the surface of the genetic carrier;
(d) recovering the genetic carrier displaying on its surface the biocatalyst; and
(e) performing the bioconversion reaction using the genetic carrier displaying on its surface the biocatalyst.

59. The method according to claim 57 or 58, wherein the spore is derived from a spore-forming Gram negative bacterium including Myxococcus, a spore-forming Gram positive bacterium including Bacillus, a spore-forming Actionmycete, a spore-forming yeast or a spore-forming fungus.

60. The method according to claim 59, wherein the spore is derived from a spore-forming Gram positive bacterium.

61. The method according to claim 60, wherein the spore is derived from Bacillus.

62. The method according to claim 57, wherein the display motif is derived from a spore coat protein.

63. The method according to claim 62, wherein the spore coat protein is selected from the group consisting of CotA, CotB, CotC, CotD, CotE, CotF, CotG, CotH, CotJA, CotJC, CotK, CotL, CotM, CotS, CotT, CotV, CotW, CotX, CotY, CotZ, SpoIVA, SspoVID and SodA.

64. The method according to claim 62, wherein the spore coat protein is a modified form of one selected from the group consisting of CotA, CotB, CotC, CotD, CotE, CotF, CotG, CotH, CotJA, CotJC, CotK, CotL, CotM, CotS, CotT, CotV, CotW, CotX, CotY, CotZ, SpoIVA, SspoVID and SodA, in which the modified form has a more compatibility for spore surface display relative to wild type spore coat protein.

65. The method according to claims 64, wherein the modification of the spore coat protein is obtained by mutating a gene encoding the spore coat protein according to a method selected from the group consisting of DNA shuffling method, StEP method, RPR method, molecular breeding method, ITCHY method, error-prone PCR, point mutagenesis, nucleotide mutagenesis, combinatorial cassette mutagenesis and other suitable random mutagenesis.

66. The method according to claim 63 or 64, wherein the spore coat protein is CotE or CotG.

67. The method according to claim 57, wherein the surface motif is derived from randomly-synthesized peptides.

68. The method according to claim 57, wherein the surface motif is a peptide or polypeptide selected from a natural-occurring random library.

69. The method according to claim 57 or 58, wherein the biocatalyst is selected from the group consisting of a hydrolase, an oxidoreductase, a transferase, a lyase, an isomerase and a ligase.

70. The method according to claim 69, wherein the biocatalyst is a transferase.

71. The method according to claim 70, wherein the transferase is an enzyme catalyzing transglycosylation.

72. The method according to claim 71, wherein the enzyme catalyzing transglycosylation is β-galactosidase, levansucrase, dextransucrase, inulosucrase, glycogen synthase, chitin synthease, starch synthease, cyclomaltodextrin glucanotransferase or 4-α-glucanotransferase.

73. The method according to claim 57, wherein the fusion form of the display motif and the biocatalyst has an order of the display motif—the biocatalyst or the biocatalyst—the display motif.

74. The method according to claim 57 or 58, wherein the biocatalysts displayed on spore surface are covalently crosslinked.

75. The method according to claim 57 or 58, wherein the biocatalyst exhibits one or more stability selected from the group consisting of thermal stability, pH stability, a resistance to organic solvent, stability to high-concentrated salt and stability to dry, in which the stability of the biocatalyst is enhanced compared to a free biocatalyst.

76. The method according to claim 57 or 58, wherein the spore exhibits lower protease activity or no protease activity.

77. The method according to claim 57 or 58, wherein the spore is non-reproductive one.

78. The method according to claim 58, wherein the virus is a bacteriophage.

79. The method according to claim 58, wherein the biocatalyst is modified one by virtue of: (i) deleting a portion of amino acids of the biocatalyst; (ii) fusing oligopeptide or polypeptide, which enhances noncovalent bond between the biocatalyst and a surface protein of the spore or virus, to the biocatalyst; (iii) subjecting the biocatalyst to site-directed mutagenesis; or (iv) subjecting the biocatalyst to random mutagenesis.

80. The method according to claim 79, wherein the biocatalyst modified by deleting a portion of amino acids is prepared by deleting ionic amino acids from N-terminal sequence of the biocatalyst.

81. The method according to claim 79, wherein the biocatalyst modified is prepared by fusing cationic peptide to the biocatalyst.

82. The method according to claim 58, wherein the spore or virus is modified by virtue of: (i) fusing oligopeptide or polypeptide, which enhances noncovalent bond between the biocatalyst and a surface protein of the spore or virus, to its surface protein; (ii) subjecting the surface protein to site-directed mutagenesis; or (iii) subjecting the surface protein to random mutagenesis.

83. The method according to claim 58, wherein the biocatalyst has covalent bonds (i) between spore or virus surface and the biocatalyst; or (ii) between the biocatalysts.

84. The method according to claim 83, wherein the covalent bond is formed by a chemical method including glutaraldehyde treatment, a physical method including ultraviolet treatment, or a biochemical method including enzyme treatment to allow the formation of covalent bond.
